(19) United States
(12) Patent Application Publication
(10) Pub. No.: US 2009/0076156 A1
(43) Pub. Date: Mar. 19, 2009

(54) USE OF A NORADRENERGIC AGONIST, E.G. GUANFACINE, FOR THE TREATMENT OF COGNITIVE DISORDERS

(76) Inventors: Masud Husain, London (GB); Andrew David Parton, Uxbridge (GB); Paresh Arjun Malhotra, London (GB)

Correspondence Address: FROST BROWN TODD, LLC, 2200 PNC CENTER, 201 E. FIFTH STREET, CINCINNATI, OH, 45202, US

(21) Appl. No.: 11/996,226

(22) PCT Filed: Jul. 18, 2006
(86) PCT No.: PCT/GB06/02669

§ 371 Date: Sep. 25, 2008

(30) Foreign Application Priority Data
Jul. 18, 2005 (GB) 0514687.3
Aug. 9, 2005 (GB) 0516373.8

(51) Int. Cl. A61K 31/165; C07C 233/11; A61P 25/28

(52) U.S. Cl. 514/617; 564/182

(57) ABSTRACT

The present invention relates to the use of a noradrenergic agonist for the treatment of a patient with a cognitive disorder resulting from acquired brain damage, wherein the cognitive disorder includes, for example, hemi-spatial neglect.

The invention relates generally to the field of therapies for ameliorating the adverse effects of brain damage. The invention particularly relates to the use of a noradrenergic agonist in the treatment of cognitive disorders resulting from brain damage, particularly in the treatment of cognitive disorders resulting from acquired brain damage, and more particularly in the treatment of hemi-spatial neglect.

Brain damage can result from a wide variety of causes and have a wide variety of effects. One cause of brain damage is stroke, which has a prevalent occurrence in the human population. The debilitating effects of stroke vary according to the severity of the damage caused to the brain. Common disabilities that may result from a stroke include hemiplegia (paralysis on one side of the body), and hemiparesis (one-sided weakness). Stroke may cause problems with thinking, awareness, attention, learning, judgment, and memory in the patient. In addition, stroke survivors often have problems understanding or forming speech.

Hemi-spatial neglect (often also referred to as visual neglect, spatial neglect or unilateral neglect) is a common disorder of space exploration following right-hemisphere stroke, although it can also occur after left-hemisphere stroke (De Renzi, E., Disorders of space exploration and cognition., ed. Wiley, New York, 1982). Hemi-spatial neglect can also occur after other causes of brain damage. Patients suffering from hemi-spatial neglect often fail to search contralesional space (i.e., left side of space for right-hemisphere stroke patients) in everyday life.

There are a number of identifiable defective cognitive components which have been suggested as playing a role in hemi-spatial neglect. These include an impaired representation of space, which may occur in multiple frames of reference (e.g. retinoptic, head-centred, trunk-centred) or may be specific to near or far space. Also, a directional motor impairment, with patients experiencing difficulty in initiating or programming contralesional movements, may contribute to hemi-spatial neglect (Parton, A. et al., Journal of Neurology, Neurosurgery & Psychiatry 75, 13-21 (2004); Parton, A. et al., ACNR 4, 17-18 (2004)).

In addition, recent research has demonstrated deficits in attentional capacity (Husain, M. et al., Nature 385, 154-6 (1997); Duncan, J., et al., Journal of Experimental Psychology: General 128, 450-478 (1999); Robertson, IH. Neuroimage 14:S85-90 (2001); Husain M, Rorden C. Nat Rev Neurosci.4:26-36 (2003)) and sustained attention (Robertson, I. H. et al., Nature 395, 169-72 (1998); Robertson, I. H. & Garavan, H. Vigilant Attention. in The Cognitive Neurosciences III (ed. Gazzaniga, M. S.) 631-640, MIT press, Cambridge, (2004)) in some patients with hemi-spatial neglect. However, the role, if any, of non-spatial deficits (such as impaired sustained attention) in contributing to the spatial deficit in hemi-spatial neglect is far from clear. As such, the clinical definition of hemi-spatial neglect is restricted to the impaired ability of a patient to search a particular region of space (left space for right-hemisphere stroke patients). Similarly, tests for identifying patients with hemi-spatial neglect focus solely on such contralesional spatial deficits. Therefore it cannot be expected that treatments effective for non-spatial cognitive deficits will also be effective for hemi-spatial neglect.

Current treatments for ameliorating the effects of hemi-spatial neglect include rehabilitation methods based on systematic visual search training, prism adaptation, limb activation training, and physiotherapeutic methods (Parton, A. et al., Journal of Neurology, Neurosurgery & Psychiatry 75, 13-21 (2004); Parton, A. et al., ACNR, 4: 17-18 (2004)).

Initial attempts to rehabilitate hemi-spatial neglect encouraged right-hemisphere stroke patients to direct their gaze leftwards. But although these approaches showed some success in reducing hemi-spatial neglect within a particular task (e.g., reading), patients typically demonstrated little generalisation of their improved scanning behaviour to tasks outside of the training environment (Robertson, I. H. et al, Unilateral Neglect. Clinical and Experimental Studies. Hove: Lawrence Erlbaum, (1993)). Recently researchers have attempted to develop techniques that produce an automatic change in behaviour, without relying on patients adopting a new control strategy to look leftwards. The approach regarded as the most promising involves prism adaptation, using lenses that induce a rightward horizontal displacement of patients' visual fields (Rosetti, Y. et al., Nature 395 (6698): 166-9 (1998)). Recent studies have suggested that the after-effects of simple prism adaptation treatment may result in a long-lasting amelioration of hemi-spatial neglect that generalises across a wide range of deficits (Frassinetti, F. et al., Brain 125(Pt 3): 608-23 (2002)). However, further work is required to understand the mechanisms underlying such improvement, and to establish the extent of its effectiveness.

Limb activation (requiring patients to actively use their contralesional hand), caloric stimulation (involving stimulation of inner ear balance organs by administering cold or hot water in an ear) and neck vibration (to simulate head turning) have all also been used with some limited success. However, most of these behavioural techniques are not practicable and it remains unclear exactly how they mediate their effects (Parton, A. et al., Journal of Neurology, Neurosurgery & Psychiatry 75, 13-21 (2004)).

While the therapeutic techniques described above have shown some promise in treating cognitive disorders such as hemi-spatial neglect, effective drug-based therapies would have significant advantages over such behavioural treatment methods. Thus there is a need for the identification of effective alternative therapeutic treatments of cognitive disorders such as hemi-spatial neglect.

Several small studies have attempted to examine the effects of dopaminergic drugs on hemi-spatial neglect. All these investigations were motivated by using pharmacological compounds to target the dopaminergic neurotransmitter system. The results of these studies have been mixed and some of the studies did not have adequate controls. As a result, these studies do not allow a clear determination of whether dopaminergic drugs have any effect on the treatment of hemi-spatial neglect. At present, the strategy of targeting the dopaminergic system for the treatment of hemi-spatial neglect has not led to the clinical use of any dopaminergic drugs (Fleet, W. S. et al., Neurology 37, 1765-1771 (1987); Grujic, Z. et al., Neurology 51, 1395-8 (1988); Geminiani G, et al., Journal of Neurology, Neurosurgery & Psychiatry 65: 344-7 (1998); Hurford, P. et al. Arch Phys Med Rehabil 79, 346-9 (1998).

The present inventors have found that, surprisingly, cognitive enhancement using a noradrenergic neuromodulator provides an important way of ameliorating the effects of brain damage. This has particularly been shown to be the case in the amelioration of hemi-spatial neglect in patients with acquired brain damage. In particular, noradrenergic agonists have surprisingly been shown to alleviate hemi-spatial neglect in patients suffering from stroke.

In a first aspect, the invention provides the use of a noradrenergic agonist in the manufacture of a medicament for the treatment of a patient with a cognitive disorder resulting from acquired brain damage.

In a second aspect, the invention provides a method of treatment of a patient with a cognitive disorder resulting from acquired brain damage, comprising administering a therapeutically effective amount of a noradrenergic agonist to the patient.

The invention provides the potential for neuromodulatory therapy in ameliorating cognitive disorders resulting from acquired brain damage.

The inventors have found that a noradrenergic agonist enhances certain cognitive abilities which have been lost, or have deteriorated, as a result of acquired brain damage. The noradrenergic agonist may exert its positive effects via actions on prefrontal regions of the brain, and dorso-lateral pre-frontal cortex (DLPFC) in particular. Consequently, noradrenergic agonists may be used for the treatment of patients suffering from acquired brain damage to more posterior regions of the brain, and more particularly for the treatment of patients suffering from acquired brain damage, wherein the DLPFC is undamaged. In particular, the inventors have shown that a noradrenergic agonist improves space exploration in hemi-spatial neglect patients suffering from acquired brain damage. These results demonstrate that neuropharmacological targeting of undamaged brain areas can significantly boost cognitive function following acquired brain damage to other brain areas.

Noradrenergic agonists mimic and/or enhance the effects of noradrenaline on the noradrenergic neural system. Noradrenaline is known to interact with adrenoreceptors in the prefrontal cortex, which has been suggested as playing a possible role in cognitive functions such as learning and memory. Noradrenergic agonists which directly stimulate noradrenergic receptors by specifically binding to these receptors are known as direct noradrenergic agonists, or noradrenergic receptor agonists. Indirect noradrenergic agonists may act by increasing the concentration of noradrenaline at neural synapses by increasing the release of noradrenaline from nerve cell terminals, or may also reduce the re-uptake and degradation of noradrenaline (Feldman, R. S., Meyer, J. S., Quenzer, L. F., Principles of Neuropsychopharmacology. Sunderland, Mass.: Sinauer Associates, Inc., (1997)). Accordingly, the noradrenergic agonist may be a direct noradrenergic agonist, or an indirect noradrenergic agonist. The direct noradrenergic agonist may be specific for the α2 adrenoreceptor (i.e. may be a α2 noradrenergic agonist), or may be non-specific to a particular class of noradrenergic receptor. The direct noradrenergic agonist may be specific for the α2_{A} adrenoreceptor (i.e. may be a α2_{A} noradrenergic agonist), or may be non-specific to a particular subtype of α2 noradrenergic receptor (e.g. clonidine and dexmedetomidine are non-specific α2 adrenoreceptor agonists which act on all types of α2 adrenoreceptor, including the α2_{A} subtype). As an example of a direct noradrenergic agonist, guanfacine is a α2 noradrenergic agonist which has been suggested as having greater selectivity for the α2_{A} receptor (Uhlen S and Wikberg J E, European Journal of Pharmacology 202; 235-43 (1991)).

Guanfacine (N-(Aminoiminomethyl)-2,6-dichlorobenzeneacetamide; Tenex™, commercially available from IDIS Pharmaceuticals Ltd. (UK)) is a known α2 noradrenergic agonist (with evidence suggesting greater selectivity for α2_{A} noradrenergic receptors) originally identified for its effects as an antihypertensive. Guanfacine is often administered orally as guanfacine hydrochloride (N-amidino-2-(2,6-dichlorophenyl) acetamide hydrochloride). The chemical structure of guanfacine is described in U.S. Pat. No. 3,632,645.

In addition to its role as an antihypertensive, guanfacine has also been shown to enhance working memory and reduce distractability in healthy monkeys, most likely via its actions on dorsolateral prefrontal cortex (DLPFC) (Arnsten, A. F. & Contant, T. A., Psychopharmacology (Berl) 108, 159-169 (1992); Amsten, A. F. T. & Robbins, T. W., in Principles of Frontal Lobe Function (eds. Stuss, D. T. & Knight, R. T.) (2002)). Consequently guanfacine has been investigated for its potential use in the treatment of Attention Deficit Hyperactivity Disorder (ADHD).

ADHD is defined by age-inappropriate symptoms of a combination of hyperactivity, inattentiveness and impulsivity. ADHD may be a consequence of abnormal functioning of brain structures such as the frontal lobe and basal ganglia, and some research studies have suggested that certain brain regions in patients with ADHD may be reduced in volume (Castellanos F X, et al., Nat Rev Neurosci. 3:617-628 (2002); Durston S., Ment Retard Dev Disabil Res Rev. 9:184-195 (2003)). Until now there has been no indication that guanfacine may be used to treat cognitive disorders, such as hemi-spatial neglect, which result from acquired brain damage. The causes of ADHD, which begin in childhood, are so far removed from the causes of cognitive disorders such as hemi-spatial neglect (often caused by brain damage acquired in adulthood, most often involving more posterior brain regions) that it was not thought that treatment methods used to treat ADHD may be used to treat cognitive disorders such as hemi-spatial neglect.

In contrast to the use of guanfacine in the treatment of ADHD, the present invention is directed to the use of a noradrenergic agonist in the treatment of cognitive disorders resulting from acquired brain damage. The term “acquired brain damage”, when used herein, refers to a non-developmental impairment of neural function suffered to the brain, often as a result of injury or the progression of a neurodegenerative disease. In particular, the present invention is particularly applicable in the treatment of adult patients suffering from acquired brain damage. In these cases, the adult brain has often developed without any significant developmental abnormalities, and neural function in particular regions of the brain becomes impaired by brain damage acquired during adulthood. The term “adult” refers to post-pubescent individuals whose brains have developed to the stage where no major developmental changes have yet to take place. The term “adulthood” refers to the time after which an individual's brain has reached a mature anatomical stage, and when an individual has reached a mature anatomical stage, which generally occurs by around the age of 18.

Noradrenergic agonists may be used in the treatment of other disorders resulting from acquired brain damage, such as defects in speech formation, executive functions (e.g., disorders of planning or strategy, cognitive control, self-monitoring, flexible switching (Shallice, T (1998), From Neuropsychology to Mental Structure. Cambridge University Press, Cambridge, UK)), control of limb movement and memory. Noradrenergic agonists may act via the prefrontal brain regions and therefore may be used to treat cognitive disorders resulting from acquired brain damage that are ameliorated by the effects of noradrenergic agonists on these regions. It has not previously been suggested that disorders resulting from acquired neural damage may be treatable through the improved effects caused by a noradrenergic agonist.

The extent of brain damage resulting from causes as described herein may be determined by known methods available to one skilled in the art. For example, clinical brain scans may be used to identify focal brain lesions in a patient. Neuropsychological testing and neurological assessment may also be used to reveal the extent of brain damage in a patient. Whilst it is stated that the noradrenergic agonists of the invention may be used in the treatment of cognitive disorders resulting from acquired brain damage, it is acknowledged that the extent of acquired brain damage can vary considerably. The size of focal brain lesions resulting from causes such as stroke and head injury is heterogeneous. Moreover, brain scans may not reveal the entire extent of brain damage in patients with acquired brain damage resulting from causes such as stroke and head injury, or in patients with acquired brain damage resulting from causes such as the neurodegenerative conditions. In such cases, neuropsychological testing and neurological assessment may be more useful in defining the extent of brain damage. Thus it will be appreciated that the present invention may be applied in the treatment of patients in which the size and extent of brain damage varies.

The present invention may be applied in those cases where the prefrontal regions of a patient's brain are undamaged to the extent that a noradrenergic agonist can exert its effect on the prefrontal regions. As such, a noradrenergic agonist may still exert a beneficial effect in the treatment of cognitive disorders in cases where prefrontal regions have sustained some damage.

Although the invention is primarily described herein in relation to the treatment of cognitive disorders resulting from brain damage caused by stroke, the invention may equally be applied in those cases where brain damage has been caused other than by stroke. Causes of acquired brain damage are well known to the person skilled in the art, and include damage resulting from neurodegenerative conditions (including causes of dementia such as Alzheimer's disease, vascular dementia, frontotemporal dementia and Cortical Lewy Body Disease; Parkinson's Disease; and Huntington's Disease) hereditary disease, physical damage sustained to the head (traumatic brain injury), lack of blood supply to the brain (including as a result of stroke or vascular dementia), damage caused by tumour growth, neuroinflammatory conditions (e.g. Multiple Sclerosis), and other causes.

A noradrenergic agonist, preferably guanfacine, may be provided to a patient in any of a number of formulations. Indeed, a noradrenergic agonist may be administered by any appropriate route, for example by the oral (including buccal or sublingual), nasal or parenteral (including subcutaneous, intramuscular, intravenous or intradermal) route. Such formulations may be prepared by any method known in the art of pharmacy, for example by bringing into association the active ingredient (noradrenergic agonist) with a pharmaceutically acceptable carrier or excipient. Suitable pharmaceutically acceptable carriers or excipients are well known to those skilled in the art.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The composition or medicament of the invention may be presented in unit dose form containing a predetermined amount of active ingredient per dose. The active ingredient is a noradrenergic agonist. Such a unit dose may be adapted to provide from 0.1-3.0 mg/day, of noradrenergic agonist (including from 0.5-2.5 mg/day, from 1.0-2.0 mg/day, or 2.0 mg/day), or from 1-50 μg/kg body weight (including from 5-45 μg/kg body weight, from 10-40 μg/kg body weight, from 15-35 μg/kg body weight, from 20-30 μg/kg body weight, or 29 μg/kg body weight).

The medicament or composition of the invention may be provided as a daily dose, or may be provided as a single dose for another specified period of time. For example, a single dose of a noradrenergic agonist may be provided once a week, once every two weeks, once every three weeks, once every four weeks, or once for any intervening time period. Since a noradrenergic agonist may exert a long term positive effect on patients suffering from a cognitive disorder resulting from acquired brain damage, the noradrenergic agonist may be provided as part of a dosage regimen designed in accordance with the time period over which the noradrenergic agonist exerts its effect. Such a dosage regimen will be easily determined by the person skilled in the art.

In those embodiments where guanfacine is the chosen noradrenergic agonist, the composition is preferably administered to a patient in the form of a suspension. Additionally, guanfacine is preferably provided to a patient at a dose in the range of about 1-50 μg/kg body weight.

Such doses can be provided in a single dose or as a number of discrete doses. The ultimate dose will of course depend on the condition being treated, the route of administration and the age, weight and condition of the patient and will be at the doctor's discretion. In addition, the range of effective dosages of each particular noradrenergic agonist may vary, and may be determined by routine experimentation known to the person skilled in the art. Preferred unit dosage formulations are those containing a daily dose or sub-dose, as herein above recited, or an appropriate fraction thereof, of a noradrenergic agonist.

The noradrenergic agonist is provided in a therapeutically effective amount. This may vary according to the severity of the cognitive defect, the age and relative health of the patient, the potency of the compound, and other factors.

It should be understood that in addition to the noradrenergic agonists particularly mentioned above, the formulations may also include other agents conventional in the art having regard to the type of formulation in question, for example those suitable for oral administration may include flavouring agents.

The invention is concerned with the treatment of mammalian patients, and preferably human patients.

Preferred features of each aspect of the invention are as for each of the other aspects mutatis mutandis. The prior art documents mentioned herein are incorporated by the fullest extent permitted by law.

### EXAMPLE

The invention will now be described with reference to the following non-limiting example. Reference is made to the accompanying drawings in which:

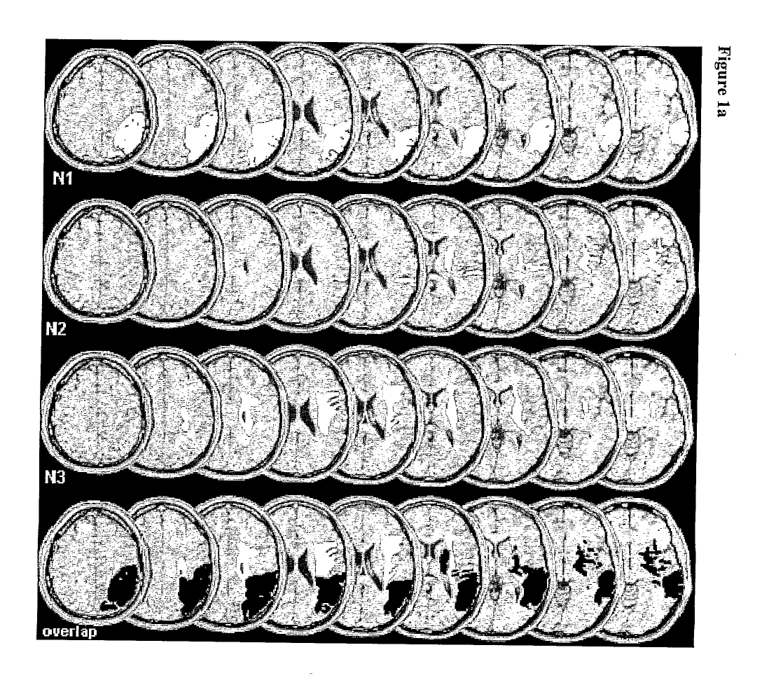

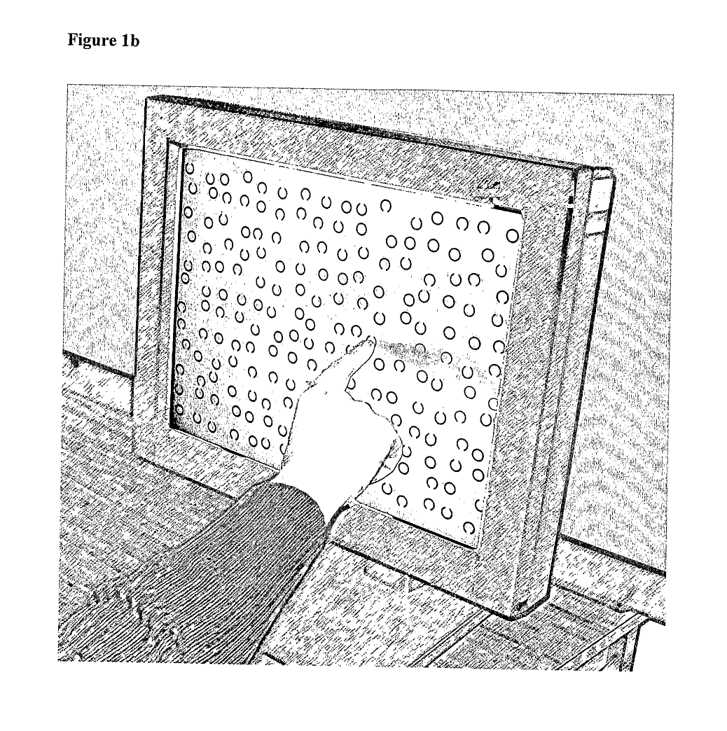

FIG. 1(a) shows lesion reconstructions for three patients (N1-N3) plotted from MRI (magnetic resonance imaging) acquisitions. The overlap figure (at the bottom) shows in white the frontal regions involved (damaged) in patient N3 that were not affected in N1 and N2; black regions mark the those parietal areas common to N1 and N2 as well as the insula and subcortical areas common to N2 and N3.

FIG. 1(b) shows the touchscreen system used to measure visual space exploration. In this embodiment the participant is searching for targets which are circles with gaps at the top. In this embodiment, distractors are whole circles and those with gaps at the bottom.

FIG. 1(c) shows the number of targets found by patients N1-N3 on the visual space exploration task, with performance following guanfacine shown in dark grey. Patient N1 was randomized to receive guanfacine between the first and second testing session on Day 14, while N2 and N3 were given the drug on Day 7. Following guanfacine, both N1 and N2 found more targets on the space exploration task but patient N3 showed no such change.

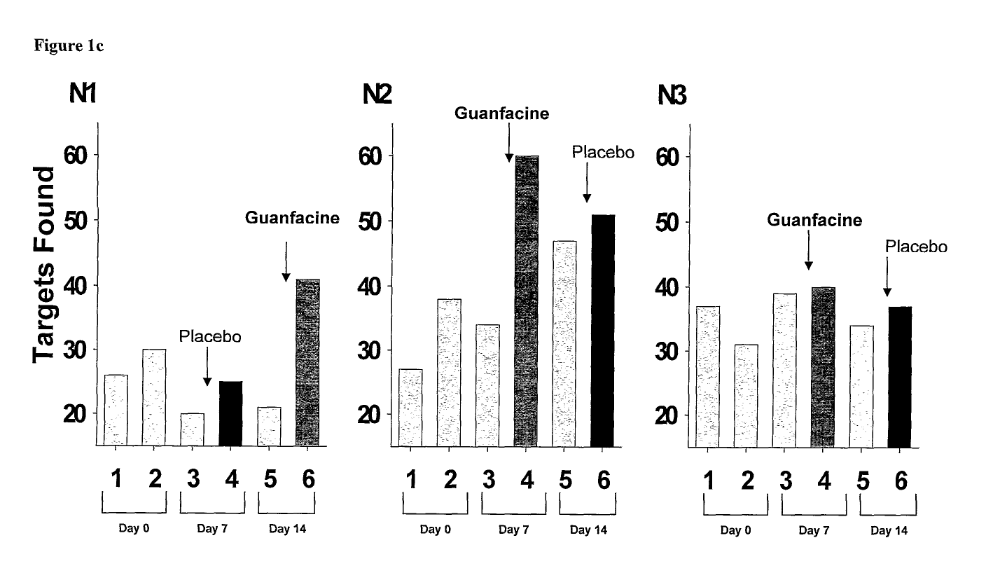

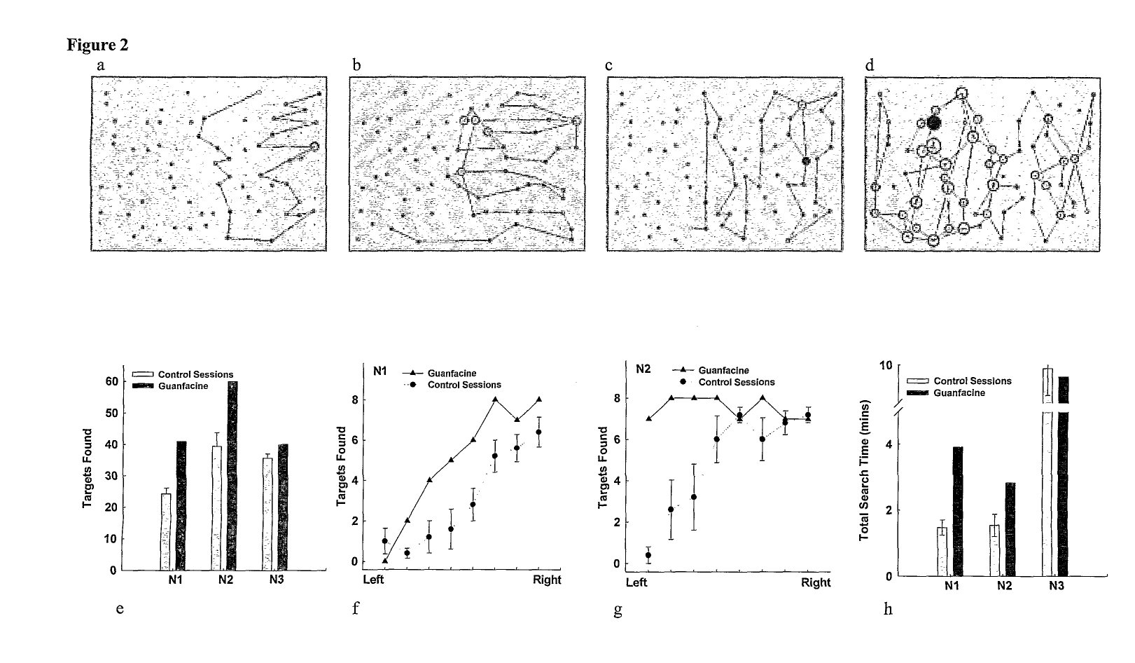

FIG. 2(a)-(c) illustrate examples of space exploration in Patient N2 in three test sessions prior to the administration of guanfacine.

FIG. 2(d) illustrates space exploration following the administration of guanfacine to Patient N2.

FIG. 2(e) shows the total number of targets found following administration of guanfacine. Both N1 and N2 showed a significant increase in number of targets (max=64) found compared to the five control sessions.

FIG. 2(f),(g) illustrate the number of individual targets found across space for N1 and N2. Space across the array (left-to-right) has been binned into 8 columns, each with a maximum of 8 targets (error bars=SEM). There was a leftward shift in the search functions for both these patients following administration of guanfacine.

FIG. 2(h) shows the total search time on space exploration task. Both N1 and N2 also showed a significant increase in time spent on the space exploration task following guanfacine compared to the five control sessions.

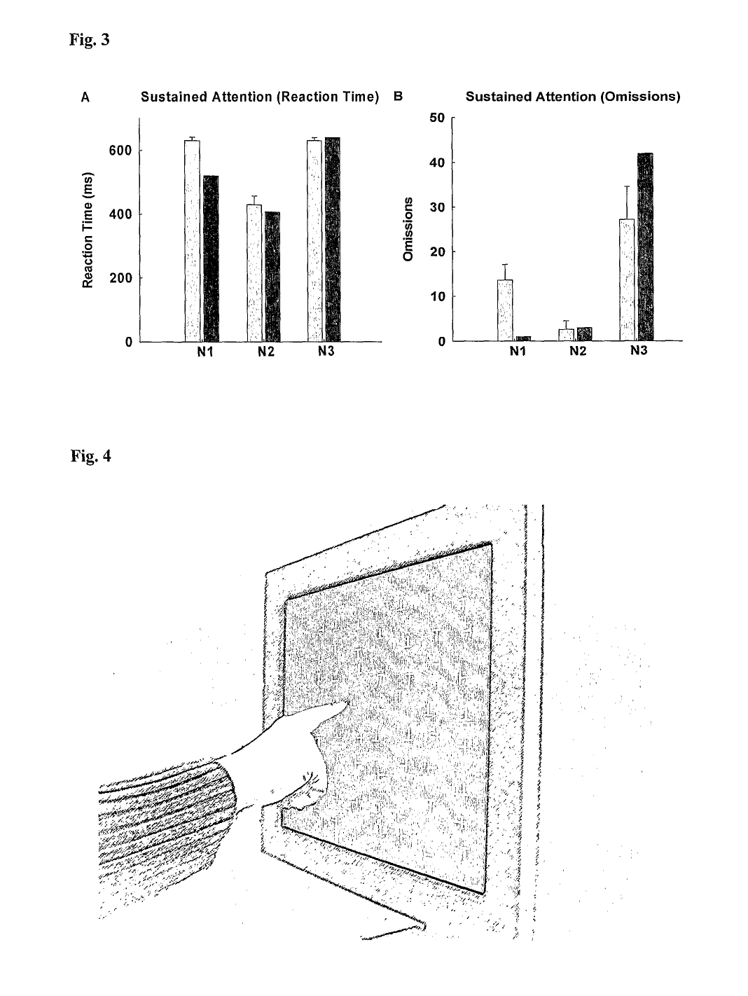

FIG. 3 shows the effects of Guanfacine on a test of Sustained Attention. Columns in light grey represent mean performance in all 5 sessions where active drug was not given (Errors bars=SEM). Columns in dark grey show performance following administration of guanfacine.

FIG. 4 illustrates a Single Target Visual Search task. The patient is asked to find and touch the letter ‘L’ among distractors.

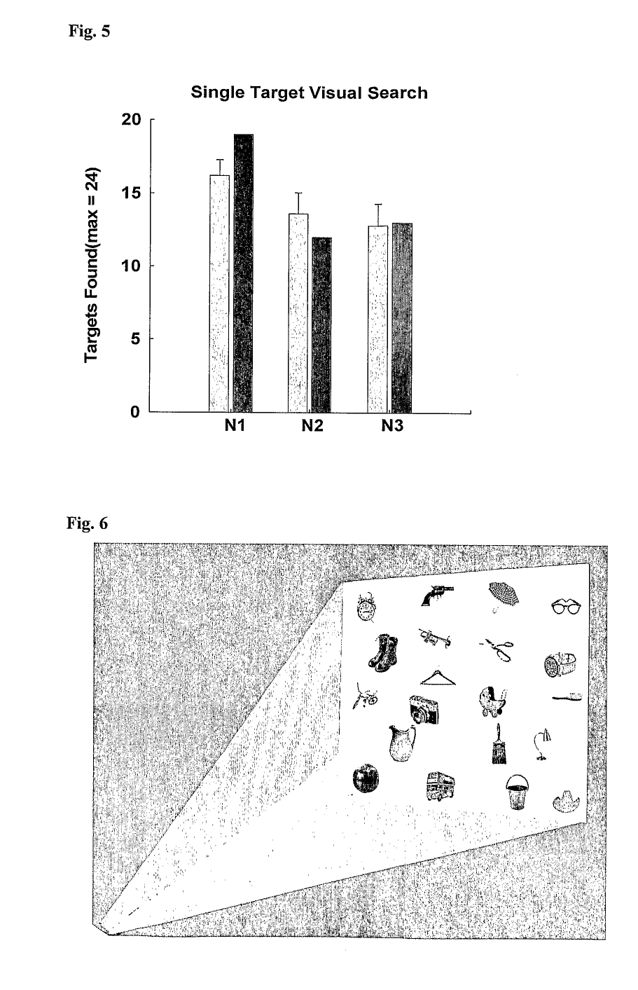

FIG. 5 illustrates the effects of Guanfacine on Singleton Target Visual Search. Columns in light grey show mean performance (and SE) in all 5 sessions where active drug was not given. Columns in dark grey represent performance following guanfacine.

FIG. 6 illustrates a test for Naming Objects in a Projected Array. Participants were exposed to a large array of twenty images of everyday objects projected onto a screen for 15 seconds and asked to name them as quickly as possible.

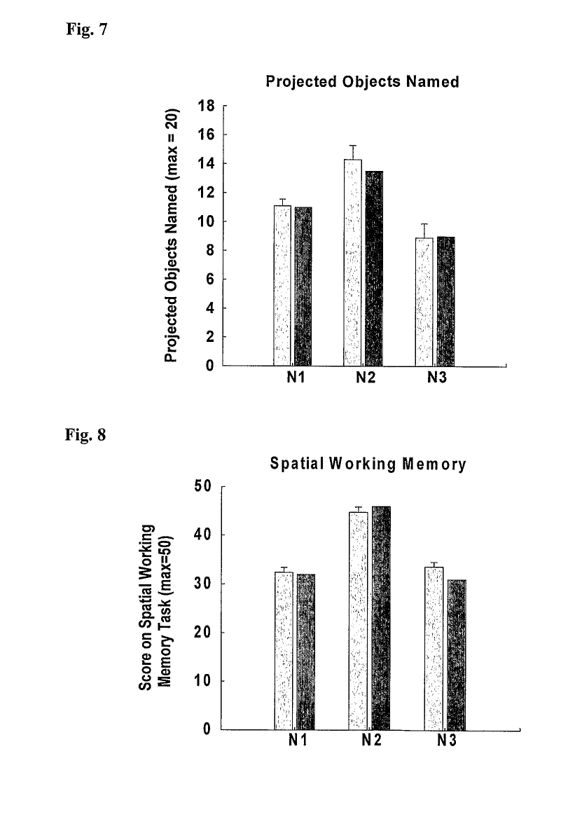

FIG. 7 shows results from the Naming Objects in a Projected Array task. Mean number of objects named from two consecutively projected arrays (maximum=20). Columns in light grey show mean performance (and SE) in all 5 sessions where active drug was not given. Columns in dark grey give the performance following guanfacine.

FIG. 8 illustrates performance on a vertical spatial working memory task. Light grey bars represent mean performance in all 5 sessions where active drug was not given (Errors bars=SEM). Dark grey bars show performance following guanfacine.

A cross-over design was used that allowed a control for placebo effects and, crucially, enabled the detection of significant changes within single individuals, extending the classical neuropsychological single-case approach to pharmacological intervention.

The effects of a noradrenergic agonist were investigated by performing detailed assessments of space exploration, visual search for single items, sustained attention and spatial working memory in patients with sustained right-hemisphere brain damage (over 23 months after stroke) who had given informed consent to participate in a proof-of-principle study. All of the patients had chronic neglect, thereby minimizing concerns about improvements in baseline neglect severity or spontaneous recovery during the course of investigation. Patient N1 had involvement (damage) of parietal and temporal cortex, while patient N2's lesion involved parts of parietal and temporal cortex as well as the insula and subcortical frontal regions. By contrast, the third patient (N3) had extensive prefrontal damage involving DLPFC and underlying subcortical white matter as well as the insula (FIG. 1a).

### 1. Patient Details:

N1 was a 73 year old man tested 5 years after his right hemisphere infarct. He had no residual weakness or sensory disturbance but continued to display left-sided extinction on examination and visual neglect in everyday life. On the first testing session of Day 0 he bisected a mean of 2 cm to the right of the true midpoint on three 18 cm horizontal lines (Manning, L. et al., Neuropsychologia 28, 647-55 (1990)), and scored 30/35 on the Bells cancellation task (Gauthier, L. et al., International Journal of Clinical Neuropsychology 11, 49-54 (1989)).

Patient N2 was a 65 year old man who suffered a right intracerebral haemorrhage 6 years prior to the study. He originally presented with left hemiparesis, homonymous hemianopia and neglect. He did not recover fully from his weakness or field deficit, and continued to show signs of left neglect in daily life. On the first test of Day 0 he bisected a mean of 1.4 cm to the right of the true midpoint on three 18 cm horizontal lines, and scored 19/35 on Bells cancellation.

N3 was a 59 year old man who suffered a large right hemisphere haemorrhage 23 months before taking part in the study. He presented with a severe left hemiparesis and reduced tactile sensation which did not recover, as well as left-sided visual extinction on examination and neglect in daily activities, both of which also continued to persist. On the first testing session of Day 0 he scored 29/35 on the Bells cancellation task and, like many neglect patients with frontal involvement (Binder, J. et al., Arch Neurol 49, 1187-94 (1992), he did not show leftward neglect on line bisection

All participants were right-handed. Informed consent was obtained using methods approved by the Riverside Research Committee, Charing Cross Hospital, London.

Each patient was tested on a battery of experimental and clinical tests on six separate sessions. On Day 0 patients were assessed twice, 90 minutes apart, without any intervention. A week later, on Day 7, patients were tested again with the same battery and then given either a single dose of oral guanfacine (29 mcg/kg body weight) or placebo. Both patients and investigators were blinded to whether active drug or placebo was administered. Patients were re-tested on the behavioural battery 90 minutes after drug/placebo (peak time of action of guanfacine). A washout period of a week was used (mean half-life of guanfacine=17 hours) before participants were tested again on Day 14. They underwent the same procedures as on Day 7, but this time they were given the alternative to the drug/placebo they had received previously. Thus each patient acted as their own control.

### 2. Tests Designed to Assess the Effects of Guanfacine:

The battery of tests used to investigate the role of noradrenergic agonists in the treatment of impaired cognitive functions included traditional tests for neglect (line bisection and cancellation) as well as experimental paradigms designed to assess visual space exploration, singleton visual search, naming objects in a projected array, sustained attention, and spatial working memory. For the measure of space exploration a new self-ordered search test was used which required participants to find as many targets as they could on a large touchscreen display (FIG. 1b). In total there were 64 targets embedded among 128 distractors. Patients indicated they had found a target by touching it but, unlike traditional clinical cancellation tasks, no visible mark was left and, importantly, no time limit was imposed. Both these features of the paradigm mean that it more closely resembles search in naturalistic settings (where no marks are left on locations that have been visited), while at the same time allowing measures to be obtained that can be related to previous tests of multi-target search in visual neglect.

All the computerised tasks described below were prepared and run in E-Prime psychological software (Psychology Tools Inc., Pittsburgh, USA).

### 2.1 Visual Space Exploration

192 black elements (64 targets and 128 non-targets) were presented against a uniform grey background on a 40×32 cm touchscreen (NEC MultiSync LCD2080UX with capacitive touch-screen technology—Mass Multimedia, Inc., Colorado Springs, USA). Displays contained three types of circular element (13 mm in diameter): (i) whole circles, (ii) circles with a small gap (10 pixel) in the top and (iii) circles with a small gap in the bottom. The targets for each participant were either circles with a gap in the bottom, or the top. The target was always the same type for each individual across testing sessions. Non-targets (whole circles and the other type of circle with a gap) are referred to as distractors.

Targets and distractors were arranged into 16 columns and 12 rows, spaced evenly over the area of the screen. Every column contained 4 elements of each the 3 stimulus types, positioned pseudo-randomly. A jitter (±20 pixels or approximately 6 mm) was applied independently to the co-ordinates of each element to reduce the apparent structure of the displays.

Participants sat at a comfortable reaching distance (approximately 57 cm) from the screen and were instructed to touch as many individual targets as they could find. Unlike traditional cancellation tasks, no visible changes occurred on the display when targets were touched. Thus there were no new marks left to act as reminders of which locations had already been explored. Hence the term ‘invisible cancellation’ has been applied to this type of space exploration task (Wojciulik, E. et al., J Neurol Neurosurg Psychiatry 75, 1356-8 (2004)).

Patients were instructed to avoid retouching targets they had already touched. There was no time limit to the task which ended when participants told the experimenter that they could find no more targets. Experiments were run using a laptop computer (Sony F809K). This touchscreen system has been validated on a sample of 22 right hemisphere stroke patients as a sensitive method for detecting neglect. Moreover, the general method of ‘invisible cancellation’ has also been used to study neglect using other response techniques (Wojciulik, E. et al., J Neurol Neurosurg Psychiatry 75, 1356-8 (2004); Mannan, S. et al., Journal of Cognitive Neuroscience 17, 340-354 (2005)). Both patient N1 and patient N2 showed a significant increase in the number of individual targets found on this task, as described above (FIG. 2).

### 2.2 Sustained Attention

Participants were tested using a laptop computer (Toshiba Satellite Pro XP 22), seated at a distance of approximately 50 cm from the laptop screen (measuring 28.5 cm×21.5 cm). They were exposed to the infrequent appearance of a central black circle (8 mm diameter) on a uniform grey background and asked to respond as quickly as possible by pressing the central button on a response box (PST Serial Response Box, Psychology Tools Inc.) The circle was presented every 1-7 seconds, remaining on the screen for 1 s. 100 stimuli were presented over a total period of approximately 8 minutes. Reaction times (within 1 second after stimulus onset) and omissions were recorded.

Patient N1 showed a significant improvement in reaction time (RT) and error rate following guanfacine (FIG. 3). Neither N2 nor N3 showed any significant change in RT or omission errors, although it should be noted that patient N2's baseline mean RT was much lower than those of the other subjects. He also made very few omission errors so there was little room for further improvement on this measure.

### 2.3 Singleton Target Visual Search

In this speeded task each patient was exposed to a display containing only one target (letter L) embedded among 63 distractors (letter Ts displayed in four different orientations from vertical: 0°, 90°, 180° and 270°). The target and distractors (both 16 mm) were white and presented on a uniform grey background on the touchscreen described above, and patients sat a distance of approximately 57 cm from the screen. The target and distractors were arranged in a pseudorandom array (FIG. 4).

Patients were instructed to look for the letter L and to touch it on the screen as soon as they found it. All trials contained a target and were terminated after 15 seconds if the patient had not touched the screen. Each patient performed 24 consecutive trials in each testing session. None of the three patients showed any significant change in the number of targets they found after receiving guanfacine (FIG. 5).

### 2.4 Naming Objects in a Projected Array

In this speeded task, participants were asked verbally to report the names of twenty objects projected onto a large screen (1.6 m×2.2 m) at a viewing distance of approximately 2.5 m. Total time for the task was 15 s. The images of objects were arranged into 4 pseudorandom columns so that 10 objects were projected on either side of the patient's midline. Images were of everyday objects or animals (FIG. 6) and when projected measured approximately 20 cm×20 cm. The images were randomly selected from 7 sets of 20 images and no set was used twice in the same testing session. Patients were tested on two image arrays per testing session. There was no significant change in the mean number of images named by each patient after taking guanfacine (FIG. 7).

### 2.5 Spatial Working Memory

A shortened version of a vertical spatial working memory task previously shown to demonstrate a deficit in patients with neglect (Malhotra, P. et al., Brain 128, 424-435 (2005)) was used.

Subjects viewed the same laptop screen as for the sustained attention task, seated at a distance of approximately 50 cm from the screen. A vertical array of ten black discs, each 1.5 cm in diameter and separated by 0.4 cm, was presented along the vertical meridian of the screen with a central fixation cross.

During each trial a sequence of discs was highlighted consecutively in a purple colour. Sequence lengths varied between 1-5 spatial locations. Each disc was highlighted for 1 s. No disc was highlighted twice during a sequence and all locations were equally likely to be part of the sequence.

Immediately after viewing a sequence, subjects were shown a 50% density random dot mask (duration 1 s) followed by the vertical array of discs with a single highlighted location. A tone was their cue to give a verbal “yes”/“no” response about whether they considered the highlighted location to have been part of the sequence they had just viewed.

There was a 50% probability on each trial that the location being probed had been part of the sequence. Subjects were free to move their eyes throughout the experiment.

Guanfacine did not significantly affect the number of correct responses made by any of the three patients (FIG. 8).

### 3. Analysis

FIG. 1c shows the number of targets found by each subject on the space exploration task in all six sessions, with performance following the noradrenergic agonist shown in dark grey. To take into account random baseline variation in neglect severity, as well as any possible systematic effects over time (e.g. practice), the effect of the noradrenergic agonist for each patient was compared against all the other five test sessions. Both patients N1 and N2 found significantly more targets on the space exploration task following administration of the noradrenergic agonist than on the other five control test sessions (FIG. 1(c), FIG. 2). N1 found 41 targets after the noradrenergic agonist compared with a mean of 24.4 (SE=1.8) in the 5 control sessions (z=4.11, p<0.0001); while N2 found 60 targets post-noradrenergic agonist compared to a mean of 39.4 (SE=4.3) in the control sessions (z=2.12, p=0.017).

FIGS. 2a, b and c show how space exploration in patient N2 was limited to the right on three control sessions prior to the noradrenergic agonist. But following the noradrenergic agonist, there was a marked shift into the previously neglected left sector of space (FIG. 2d). The positive effect of the noradrenergic agonist on leftward search is shown more formally in plots of individual targets found across space for N1 and N2 (FIGS. 2f,g). In contrast, patient N3 showed no significant increase in the total number of targets found after the noradrenergic agonist.

Interestingly, patient N2 performed better on the visual exploration task on Day 14 (one week after the noradrenergic agonist) than prior to receiving the drug (FIG. 1c). This may be attributable to a long-lasting effect of the noradrenergic agonist. For patient N2, the change in performance between first and second testing sessions on each experimental day was maximal immediately following the noradrenergic agonist, compared to Day 0 or following placebo (FIG. 1c). Furthermore, by taking into account the variation across all five non-noradrenergic agonist sessions when testing for significance the most conservative criteria was adopted for such comparisons, which assumed that N2's performance on Day 14 was independent of the effects of the noradrenergic agonist.

The beneficial effects of a noradrenergic agonist on space exploration in N1 and N2 were associated with significant increases in total search time, with a greater than two-fold rise in time spent performing the task (FIG. 2h). In patient N1 the total search time went up to 235 seconds from a mean of mean 89 s (SE 14) in the control sessions (z=4.79, p<0.0001); while in N2 it increased to 170 s from 93 s (SE 20) in the control tests (z=1.7,p<0.05). There was no alteration in the mean time required to find individual targets. For N3 there was no significant change in either total search time or mean time to find targets.

These improvements on the space exploration task observed in N1 and N2 may be due to enhanced sustained attention on the task, with guanfacine extending the duration of search and increasing the probability of finding leftward targets. Consistent with this account, Patient N1 showed a significant improvement on our independent test of sustained attention (a simple reaction time task to infrequent visual stimuli) following guanfacine (FIG. 3). He demonstrated a reduction in both reaction time and error rate following the drug (post guanfacine RT=520 ms vs. 630 ms (SE=10.8) in control tests (z=4.56,p<0.000); and error rate dropped to 1 from 13 (SE=3.5) in control trials, p=0.05, one-tailed). However, there was no such significant improvement in either of the other two patients.

Although guanfacine's mean half-life in humans is ˜17 hrs, there appears to have been a long-lasting effect of guanfacine in patient N2, who was randomized to receive the drug on Day 7 and therefore was re-tested subsequently when he received placebo on Day 14. Thus, the noradrenergic agonist may exert a positive long-term effect on patients suffering from acquired brain damage, in addition to more immediate effects.

None of the participants showed a significant benefit following the noradrenergic agonist on singleton visual search (FIGS. 4, 5) or naming objects in a projected array (FIGS. 6, 7), demonstrating that spatially lateralised bias on these speeded visual search tasks was not modulated by the noradrenergic agonist. Standard cancellation and line bisection were also not significantly altered. These traditional clinical tests may not be as sensitive to deficits of space exploration as the touchscreen system developed by the present inventors. The touchscreen space exploration task used here consists of a larger spatial array than standard clinical tests and, as noted above, visible cancellation marks are not left as reminders of locations that have already been explored. Even pen-and-paper versions of such ‘invisible’ cancellation have proven to be more sensitive than more traditional methods where visible marks are made on targets that have been found (Wojciulik, E. et al., J Neurol Neurosurg Psychiatry 75, 1356-8 (2004)). Finally, on the computerised test of spatial working memory used here, none of the patients showed a significant benefit following administration of a noradrenergic agonist (FIG. 8). Thus, there was no evidence that a noradrenergic agonist's positive effects in hemi-spatial neglect were mediated by improving spatial working memory.

Guanfacine led to significant improvements in leftward search on a space exploration task in patients with acquired brain damage. The different effects observed in the patients could not be accounted for by lesion volume since both the patients with the largest (N1) and smallest (N2) lesions showed improvement while another patient (N3), with intermediate size lesion, did not (lesion volumes: N1 94.5 cm³; N2 29.1 cm³; N3 38.5 cm³).

There appeared to be no improvement in spatially lateralised bias on time-limited search tasks or in spatial working memory, as might be expected if the effects of the noradrenergic agonist were largely mediated through improvements in sustained attention. Instead, the beneficial effects of the noradrenergic agonist in maintaining attention on task and shifting search leftwards were most prominent on a self-ordered exploration task in which there are no time limits or visible reminders of locations that have been inspected, analogous to real-world search.

### Non-cognitive Effects of Guanfacine

Blood pressure (BP) is known to be reduced by guanfacine. BP in each patient was recorded by an individual who was not one of the study investigators immediately before being drug/placebo and 90, 120 and 150 minutes later. Patient N2 showed the most substantial change following guanfacine with a decrease of >25 mmHg in systolic pressure. N1 and N2 both had decreases in diastolic blood pressure following the drug (Mean maximum decrease: systolic: 10.67 (SEM 9.2), diastolic 16 SEM (3.6)). None of the patients were symptomatic and none reported any side-effects following active drug or placebo.

1. The use of a noradrenergic agonist in the manufacture of a medicament for the treatment of a patient with a cognitive disorder resulting from acquired brain damage.

2. The use of claim 1, wherein the brain damage is acquired during adulthood.

3. The use of claim 1 or claim 2, wherein the noradrenergic agonist is a noradrenergic receptor agonist.

4. The use of claim 3, wherein the noradrenergic agonist is an alpha-2 adrenergic receptor agonist.

5. The use of claim 4, wherein the noradrenergic agonist is an alpha-2A adrenergic receptor agonist.

6. The use of any preceding claim, wherein the noradrenergic agonist is guanfacine.

7. The use of any preceding claim, wherein the medicament is formulated to provide a dose in the range of from about 1 μg/kg body weight to about 50 μg/kg body weight of noradrenergic agonist.

8. The use of claim 7, wherein the medicament is formulated to provide a dose of about 29 μg/kg body weight of noradrenergic agonist.

9. The use of any preceding claim, wherein the cognitive disorder is selected from the group consisting of dysphasia, defects in executive functions, and defects in the control of limb movement and memory.

10. The use of any one of claims 1-8, wherein the cognitive disorder is hemi-spatial neglect.

11. The use of any preceding claim, wherein the patient is a human.

12. A method of treatment of a patient with a cognitive disorder resulting from acquired brain damage, the method comprising administering a therapeutically effective amount of a noradrenergic agonist to the patient.

13. The method of claim 12, as modified by the features of any one of claims 2-11.
